(19) United States
(12) Patent Application Publication
(10) Pub. No.: US 2011/0319857 A1
(43) Pub. Date: Dec. 29, 2011

(54) STIFFENING ELEMENT AND ABSORBENT ARTICLE COMPRISING SUCH AN ELEMENT

(75) Inventors: Solgun Drevik, Molnlycke (SE); Ann Samuelsson, Lindome (SE)

(73) Assignee: SCA HYGIENE PRODUCTS AB, Goteborg (SE)

(21) Appl. No.: 13/254,756

(22) PCT Filed: Mar. 6, 2009
(86) PCT No.: PCT/SE2009/050236

§ 371 Date: Sep. 2, 2011

(51) Int. Cl. A61F 13/47

(52) U.S. Cl. 604/385.31

(57) ABSTRACT

A stiffening element for an absorbent article designed to, at least during use of the article, provide the article with a predetermined shape that enhances the fit of the article to a wearer's body. The stiffening element has a length in a longitudinal direction and a width in a transverse direction, an upper side intended to face towards a wearer during use of the article, and a lower side intended to face away from the wearer during use of the article. The stiffening element extends in the longitudinal direction over at least a part of both a front portion and a crotch portion. The stiffening element has a width at a transition between the front and crotch portions that is less than the width in the front portion. At least a part of both the upper side and the lower side of the stiffening element exhibit mechanical fastening properties.

### CROSS-REFERENCE TO PRIOR APPLICATION

This application is a §371 National Stage Application of PCT International Application No. PCT/SE2009/050236 filed Mar. 6, 2009, which is incorporated herein in its entirety.

### FIELD-OF THE INVENTION

The present disclosure relates to a stiffening element for an absorbent article, such as a sanitary napkin, a panty liner or an incontinence protector. The stiffening element is designed such as to, at least during use of the article, provide the article with a predetermined shape that enhances the fit of the article to a wearer's body. The disclosure also relates to an absorbent article including such a stiffening element.

### BACKGROUND

Absorbent articles, such as sanitary napkins, incontinence guards, panty-liners, diapers etc., are known in the art. An important function of absorbent articles is to prevent leakage of body exudates during use of the article. Generally, the article should fit well to the user and stay in place during use. This also enhances the user comfort.

With regard to at least sanitary napkins, incontinence guards and panty-liners, it is previously known to provide the article with stiff or elastic shaping elements that provide the article with a shape that improves the fitting and the ability to stay in place during use. In general, a stiff shaping element has the advantage that the shape of the article is predetermined and maintained during use. On the other hand, stiff shaping elements should be designed with particular care in order not to cause discomfort during use of the article. It is also known to provide the underside of sanitary napkins and similar absorbent articles with fastening means, such as adhesives, for attachment to the user's garments.

WO 0117474 discloses an example of an absorbent article in the form of a sanitary napkin, a panty liner or an incontinence protector, wherein the rear portion of the article includes a longitudinally extending ridge-shaped elevation forming a stiff shaping element that partially extends between the wearer's buttocks during use of the article. This provides good protection against rearward leakage.

WO 98/22061 discloses an absorbent article in the form of a sanitary napkin having stiff front and crotch portions wherein the front portion is curved and inclined upwards, towards the user, with respect to the crotch portion. Further, the article has a narrow waist in the crotch portion allowing a high stiffness without causing discomfort. The desired stiffness is achieved by e.g. including a rigid shape-retaining, spoon-shaped, plastic or metal layer inside the article. The article according to WO 98/22061 is intended to be kept securely and comfortably in position against the body of the user during use, without the need for particular attachment means.

EP 1395218 discloses an adsorbent article in the form of a sanitary towel or incontinence pad including a combined, flat stiffening and absorbent element arranged inside the article, which element gives the article in different regions a predetermined two- or three-dimensional shape (including curvature, bowl-shape and a raised part between the buttocks of the wearer) during use of the article, i.e. when the article is affected by compressive forces generated by the thighs of the wearer. In similarity to WO 98/22061, the front and crotch portions are designed to allow anchoring of the article to thigh muscle tendons, which gives the article a good fit and stability in the fitted position.

Although the known absorbent articles with stiffening elements in many cases provide for a good fit, there still remains a need to further develop this type of absorbent articles.

### SUMMARY

It is desired to provide a stiff shaping element for an absorbent article, such as a sanitary napkin, that exhibits improved properties compared to conventional stiff shaping elements. This can be achieved by the disclosed stiffening element.

A first aspect relates to a stiffening element for an absorbent article, such as a sanitary napkin, a panty liner or an incontinence protector. The stiffening element is designed such as to, at least during use of the article, provide the article with a predetermined shape that enhances the fit of the article to a wearer's body. The stiffening element has: a length in a longitudinal direction, a width in a transverse direction, an upper side intended to face a wearer during use of the article, and a lower side intended to face away from the wearer during use of the article. The stiffening element extends in a longitudinal direction over at least a part of both a front portion and a crotch portion, and the stiffening element has a width at a transition between the front and crotch portions that is less than the width in the front portion.

The stiffening element includes a material exhibiting mechanical fastening properties, wherein at least a part of both the upper side and the lower side of the stiffening element exhibits the mechanical fastening properties.

The term mechanical fastening properties as used herein refers to mechanical fastening means such as hooks, friction adhesives, clips, friction elements and combinations thereof. The fastening means are known to a person skilled in the art, and the fastening means allows for a detachable attachment. Thus, a material exhibiting mechanical fastening properties includes mechanical fastening means of the abovementioned type(s).

Because the stiffening element exhibits the mechanical fastening properties, it can be used as a separate, reusable element when arranged in association with the absorbent article. In particular embodiments, the upper side of the stiffening element is attached to a lower side of an absorbent member which will be facing the wearer during use. The lower side of the stiffening element can be attached either to the undergarments of the wearer of the article as to keep the article in place during use, or to an upper side of a secondary, lower absorption member as to form a two-part absorbent product including an upper and a lower absorption member.

The term absorption member as used herein refers to an item that includes an absorption body for absorption of body fluids. The absorption member may also include e.g. a liquid permeable top-sheet arranged on the upper side of the absorption body and a liquid-impermeable back sheet arranged on the lower side of the absorption body.

The stiffening element has the advantage that, when arranged in an absorbent article, it provides the article with a good body fit and at the same time it allows detachable attachment to the items, i.e. absorbent member(s) and user undergarment, it is attached to. Because the stiffening element provides the article with a suitable shape, it is not necessary that the absorption member contributes to the shaping of the article. Thereby it becomes possible to use a very flexible absorption member, i.e. a very flexible absorption body, which, at least with regard to the absorption member facing the wearer, improves the comfort of the absorbent article. Further, it becomes possible to use simple and non-expensive absorption members. Because of the mechanical fastening properties of the stiffening element, a soiled absorption member can easily be removed and replaced. This is useful also for two-part products since in many situations only the upper absorption member will be soiled after use of the article, in which situations only the upper absorbent member will need to be disposed and replaced. Re-use of the stiffening element is generally possible.

The possibility of attaching the absorbent article, via the stiffening element, to the garments is useful also for articles with advanced body fit, such as the one disclosed in WO 98/22061, because also such articles may move in relation to the wearer during cycling or other vigorous activities.

The possibility of attaching the stiffening element to both an upper, primary absorption member and a lower, secondary absorption member placed below the one facing the wearer is useful for providing a two-part absorbent product that, for instance, may be designed to have a larger wetting area and/or a higher absorption capacity than the corresponding one-part product (i.e. the upper absorption member). Thus, the stiffening element enables the use of a convertible article that, if the user so wishes, can be converted to a two-part product at certain occasions.

The stiffening element has a multifunction: shaping and fastening of an absorbent article. Such multifunction is generally useful for decreasing the number of components in the absorbent article which makes the manufacture more cost-effective. In the absorbent article disclosed in EP 1395218 multifunction is achieved by providing an absorbent body with a certain stiffness, i.e. stiffening (shaping) and absorbing properties are combined in the same element. However, stiffening and absorbing properties are not easily combined and such a combined element is rather costly. In contrast, the disclosed stiffening (shaping) element is a separate item in relation to the absorption element and instead of combining stiffening and absorbing properties it combines stiffening and fastening properties which are easier to combine. For instance, improvement of the fastening properties of a piece of material does not normally lead to any corresponding impairment of the stiffening properties. Further, because the absorbing function does not have to be taken into account it is not necessary that the stiffening element has a shape in the lateral plane of the article that is adapted to this function; i.e. the stiffening element secured to the lower side of the absorption member may include e.g. rectangular parts to reduce wastage in the production.

In an advantageous embodiment, the material exhibiting mechanical fastening properties also contributes significantly to the stiffness of the stiffening element. This means that the same material provides both the fastening function and, at least a great deal of, the shaping/stiffening function. This way, the structure of the stiffening element can be simplified in that the need for using particular stiffening components or layers is diminished, or even eliminated.

In a further advantageous embodiment, the material exhibiting mechanical fastening properties is a hook material having hooks protruding from at least one of the sides of the stiffening element. Mechanical fasteners in the form of hooks are known in the field of absorbent products to be suitable for attachment to textile materials such as undergarments or a non-woven material of an absorption member. In particular embodiments, the hook material includes a hook carrier layer to which layer the hooks are secured. The hook material significantly contributes to the stiffness of the stiffening member. Thereby, the hook material provides both the fastening and the stiffening/shaping functions. In a variant of this embodiment, the hook material constitutes the stiffening element. With such a design, no further materials are needed for providing the stiffening element with said functions.

In another advantageous embodiment, the material exhibiting mechanical fastening properties is a friction adhesive material. Thus, the surface of the material exhibits a stickiness that is useful for attaching the stiffening element to e.g. undergarments or a back sheet of an upper absorption member. Also friction adhesive materials are known to the person skilled in the art. In a variant of this embodiment, the friction adhesive material constitutes the stiffening element. With such a design, no further materials are needed for providing the stiffening element with said functions.

In another advantageous embodiment, the stiffening element forms a separate item that is detachably attachable to an absorbent member of the absorption article. This allows e.g. easy use and re-use of the stiffening element. Moreover, manufacture can be simplified since it is not necessary to integrate the stiffening member with other parts of the absorbent article.

In another advantageous embodiment, the width at the transition between the front and crotch portions is within the range of 15-45 mm. In particular embodiments, the stiffening element provides, at least when arranged in association with an absorbent article and during use of the article, a three-dimensional bowl-like shape in an area in the front portion. In more particular embodiments, the stiffening element has a length in the crotch portion within the range of 70-120 mm. In certain embodiments, side edges of the stiffening element, in the direction from the crotch portion in over the front portion, form an acute angle a with a line in the longitudinal direction of the stiffening element. In more certain embodiments, the stiffening element extends further in the longitudinal direction some way in over a rear portion and has a wedge-shaped cutout extending from a rear end edge of the stiffening element in a direction towards the crotch portion.

In an alternative embodiment, the stiffening element is arranged to allow through-flow of body fluids from its upper side to its lower side.

In another advantageous embodiment, the stiffening element forms part of a larger piece of material from which it can be separated. The piece of material includes a marking corresponding to a peripheral edge of the stiffening element. This allows for cost-effective manufacture since the larger piece of material may have any shape suitable for production, e.g. rectangular, and since only a marking needs to be provided. A user can easily cut out the stiffening element by herself In particular embodiments, a plurality of markings are provided corresponding to different sizes and/or geometries of the stiffening element so that a user can select the most suitable.

A second aspect relates to an absorbent article including a stiffening element of the type described above.

### BRIEF DESCRIPTION OF THE DRAWINGS

In the description of embodiments of the invention given below reference is made to the following figures, in which:

FIG. 1 shows, in a schematic, sectional side view, a first embodiment,

FIG. 2 shows the underside of an adsorbent article according to another embodiment, and

FIG. 3 shows the underside of an adsorbent article according to still another embodiment.

### DETAILED DESCRIPTION OF PREFERRED EMBODIMENTS

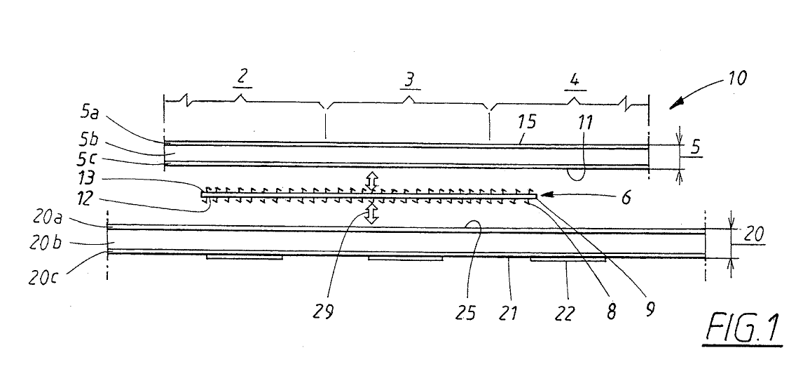

FIG. 1 shows, in a schematic, sectional, cut side view, and in a disassembled state, a first embodiment of an absorbent article 10, in this case a sanitary napkin, that includes a stiffening element 6. The absorbent article 10 and the stiffening element 6 have a longitudinal direction, i.e. the left-right direction in FIG. 1, and a transverse direction, i.e. a direction perpendicular to the plane of FIG. 1. Further, the article 10 and the stiffening element 6 have a front portion 2, a rear portion 4 and a crotch portion 3 located between the front portion 2 and the rear portion 4. The division of the article 10 and stiffening element 6 into these portions is not strict but describes, in a conventional way, the intended positioning of the article 10 and stiffening element 6 in relation to a wearer.

The absorbent article 10 shown in FIG. 1 includes an upper, primary absorbent member 5, the stiffening element 6 and a lower, secondary absorption member 20. The upper absorption member 5, which contains an upper side 15 and lower side 11, is, via a lower side 11, detachably attachable to the stiffening element 6. Similarly, the lower absorption member 20 is, via an upper side 25, detachably attachable to the stiffening element 6. Thus, the upper and lower absorbent members 5, 20 and the stiffening element 6 are separate components that are detachably attachable such as to form a two-part product with two absorption members. In FIG. 1 the absorbent article 10 is disassembled. Arrows 29 indicate that the two absorption members 5, 20 may be detachably attached to each other via the stiffening element 6.

The lower absorption member 20 has an upper side 25 intended to face towards a wearer during use of the article 10 and a lower side 21 intended to face away from the wearer during use of the article 10. The lower absorption member 20 exemplified in FIG. 1 is in principal structured in a conventional way and includes a liquid-permeable top sheet 20a, an absorbent body 20b for absorbing body fluids, and a liquid-impermeable back sheet 20c. The top sheet 20a and the back sheet 20c are interconnected around an outer edge of the absorbent body 20b such as to form a cover around the absorbent body 20b (not shown in FIG. 1).

The lower side 21 of the secondary absorption member 20 is provided with fastening means 22, for instance in the form of adhesives, for attaching the absorbent article 20 to the undergarments of a user. The fastening means 22 are optional.

Suitable materials and material combinations for forming the top sheet 20a, the absorbent body 20b, and the back sheet 20c are known. Examples of suitable materials are non-woven fabrics and perforated plastic films for the top sheet 20a; cellulose fibers, absorbing foam material and super absorbents (SAP) for the absorbent body 20b; and polyethylene film and non-woven fabrics treated with hydrophobing agents for the back sheet 20c.

Which material to choose for the top sheet 20a depends for instance on which type of mechanical fastening means the stiffening element 6 is provided with. This is further described below.

The stiffening element 6 extends longitudinally and transversely between the upper and lower absorbent members 5, 20 and has an upper side 13 facing the upper absorbent member 5 and a lower side 12 facing the lower absorbent member 20.

In the example shown in FIG. 1, the stiffening element 6 is entirely made of a hook material comprising a hook carrier layer (hook substrate) 9 and a plurality of hooks 8 secured to the hook carrier layer 9. The hook carrier layer 9 extends longitudinally and transversely. The hooks 8 protrude at least partly in a vertical direction from both sides of the hook carrier layer 9. This means that the hooks 8 protrude from both the lower and upper sides 12, 13 of the stiffening element 6. Hooks constitute one example of mechanical fasteners that are suitable for detachable attachment to textile materials. Thus, the stiffening element 6 in FIG. 1 is made of a material that exhibits mechanical fastening properties, wherein at least a part of both the upper side 13 and the lower side 12 of the stiffening element 6 exhibits the mechanical fastening properties. Other mechanical fasteners that may be used are clips or friction elements. Combinations of different types of mechanical fasteners may also be used.

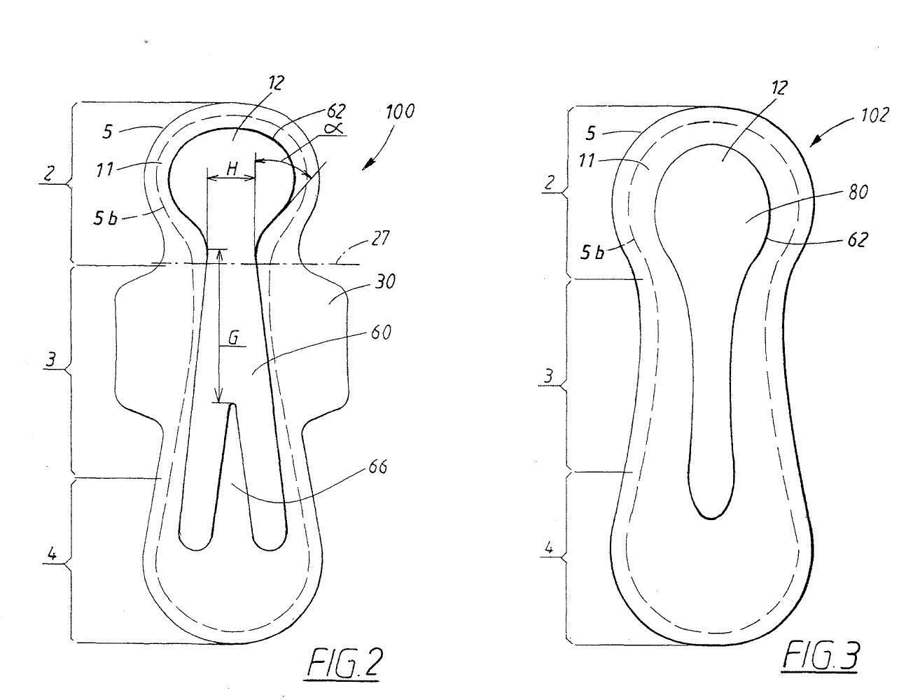

As can be seen in FIG. 1, the stiffening element 6 extends longitudinally from the front portion, over the crotch portion 3 and into the rear portion 4. This is similar to what is described in relation to FIGS. 2 and 3. The purpose of FIG. 1 is, however, only to give a schematic view of the structure of the absorbent article. Different shapes of the stiffening element are described below.

The upper absorption member 5 has a similar structure as the lower absorption member 20, i.e. it includes a liquid-permeable top sheet 5a, an absorbent body 5b for absorbing body fluids, and a back sheet 5c. However, the back sheet 5c of the upper absorption member 5 is liquid permeable to allow through-flow of body fluids towards the lower absorbent member 20. The top sheet 5a and the back sheet 5c are interconnected around an outer edge of the absorbent body 5b such as to form a cover around the absorbent body 5b (not shown in FIG. 1). In this case the back sheet 5c is made of non-woven fabrics which allows for a good attachment to the hooks 8. A back sheet made of e.g. foam or airlaid also allows for a good attachment.

If no secondary absorption member is used, i.e. if the lower side 12 of the stiffening element 6 instead is used to attach the article 10 to the undergarments of a user, it is, of course, suitable to provide the upper absorbent member 5 with a liquid-tight back sheet 5c.

In the example shown in FIG. 1, also the top sheet 20a of the lower absorbent member 20 is made of non-woven fabrics to allow for a good attachment with the hooks 8.

The stiffening element 6 can be made liquid permeable by providing it with through-holes distributed in various patterns longitudinally and transversely over the stiffening element 6. This allows through-flow of bodily fluids towards the lower absorption member 20. Alternatively, or in combination, the stiffening element 6 can be made in a liquid-permeable material.

As can be seen in FIG. 1, the secondary absorption member 20 extends further in the longitudinal direction (i.e. sideways in FIG. 1) than does the primary, upper absorption member 5. The secondary absorption member 20 also extends further in the transversal direction than does the primary, upper absorption member 5. Accordingly, the secondary absorption member 20 surrounds the primary, upper absorption member 5 as seen from above, i.e. the secondary absorption member 20 exhibits a larger area as seen in a direction towards a user of the absorbent article 20 than does the primary, upper absorption member 5. In short, in the example shown, the secondary absorption member 20 is both longer and wider than the upper absorption member 5. This is useful for increasing the leakage protection.

With regard to FIG. 1 it should be noted that it is not necessary that the back sheet 5c and the stiffening element 6 are liquid permeable to allow body fluids to be transferred to the secondary absorption member 20. If either or both of the back sheet 5c and the stiffening element 6 are liquid impermeable, body fluids may flow transversely (and longitudinally) and pass an outer edge of either or both of these components and then reach the secondary absorption member 20. At least if the back sheet 5c is liquid impermeable it is of particular importance that the secondary absorption member 20 is wider, and optionally also longer, than the primary, upper absorption member 5.

After use of the absorbent article 1, it is possible to dispose and replace only the upper absorption member 5, i.e. the lower absorption member 20 and the stiffening element 6 can be reused. To avoid unnecessary soiling of the lower absorption member 20 it may be useful to provide the upper absorption member 5 with a liquid impermeable back sheet 5b.

It may be noted that it is not necessary that the upper absorption member 5 is smaller, i.e. less wide and less long, than the lower absorption member 20. However, a smaller absorption member is sufficient in many situations and since less material is required for manufacturing a smaller absorption member it is a cost-advantage to use a smaller upper absorption member 5. The larger lower absorption member 20 can thus be regarded as an additional, safety member.

Suitable hook materials for the embodiments shown in FIGS. 1 and 2 are available as KHK0002 or CHK 00752 from 3M Company. These materials have a suitable stiffness for giving the article 10 a good shape during use and provides for a good attachment.

The hooks 8 can, of course, be chosen such as to be adapted to the intended use. That is, the hooks 8 can be adapted to interact (i.e. attach to, detach from, avoid destruction etc.) particularly well with a certain type of material. As an example, both the upper and the lower sides 13, 12 of the stiffening element 6 can include hooks 8 particularly adapted to be attached to non-woven fabrics of absorbent members. As a further example, the upper side 13 of the stiffening element 6 can include hooks 8 particularly adapted to be attached to non-woven fabrics of an (upper) absorbent member, whereas the lower side 12 can include hooks 8 particularly adapted to be attached to undergarments made of cotton. A still further example is that the stiffening element 6 is flat in its uncompressed, non-use state and that it has one side that is particularly adapted to interact with cotton textile material and another side particularly adapted to interact with a synthetic textile material, and wherein both sides are capable of interacting sufficiently well with non-woven fabrics of an absorbent member. In such a case, a user can choose which side should be facing down depending on the type of textile material of the undergarments. In particular embodiments, the stiffening element 6 is provided with an indication on the particular adaptation of its sides, for instance a color marking, so that a user easily can see how the stiffening element 6 should be positioned.

As an alternative to the hook material described above, the stiffening element 6 can, at least partly, be made of a friction adhesive material, i.e. of a material that exhibits a stickiness that can be used to mechanically fasten the stiffening element 6 to e.g. the upper absorption member 5 and the undergarments. In such a case, the back sheet 5c of the primary absorption member 5 can be made of e.g. a nonwoven material or a perforated plastic film which allows for a good attachment to the friction adhesive material.

Friction adhesive materials, which also are referred to as friction materials, should not be confused with conventional fastening adhesives. A general difference between these materials is that the pressure sensitive adhesive of conventional fastening material does provide reasonable tack, peel and shear after a bond has been initiated by putting pressure onto the system. Different to such a behavior, a friction adhesive material will mainly provide shear. This shear or friction is proportional to the force used to press the friction material onto a second surface. After release of the pressure the system will show basically no remaining tack, peel or friction.

An example of a suitable friction adhesive material is 5401 Traction Tape available from 3M Company.

Hook material and friction adhesive material may be combined in that some parts of the stiffening element 6 includes hook material and some includes friction adhesive material. For instance, one side of the stiffening element 6 can include hooks and the other friction adhesive material. A mix of hooks and friction adhesive material on the same side is also possible. It is further possible to use a material that has both properties, for instance a hook material that exhibits a stickiness.

Friction adhesive material may be involved when adapting the different sides 12, 13 of the stiffening element 6 to interact particularly well with a certain type of material as described above.

The stiffening element is sufficiently stiff for, as far as possible, preventing the absorbent article from being compressed or otherwise deformed in an uncontrolled manner during use of the article.

The stiffening element 6 should exhibit a stiffness that is higher than the material of the absorbent article 10 that surrounds the stiffening element. In relation to the embodiment described here, this means that the stiffening element 6 should be stiffer than the upper and lower absorbent members 5, 20. That the stiffness of the stiffening element 6 is higher than its surroundings has the effect that folding indications are provided along and/or around the stiffening element 6. These folding indications, together with e.g. the size and geometry of the stiffening element 6, determine which shape the article 10 will acquire during use.

In particular embodiments, the stiffening element 6 exhibits a stiffness in a dry state in the order of 1-15 N as measured according to ASTM D 4032-82.

The stiffening element 6 can have a variety of shapes and positions depending on the shape desired. Various advantageous shapes of absorbent articles are known. In any case, the stiffening element is arranged to, at least during use of the article, provide the article with a certain, predetermined shape that enhances the fit of the article to the wearer's body. A stiffening element may have a flat form before use but take a three-dimensional shape upon use of the article, i.e. when the article is affected by compressive forces generated by the thighs of the wearer. Alternatively, a stiffening element may have a three-dimensional shape already before use of the article.

In particular embodiments, the stiffening element 6 is arranged such as to, at least during use of the article, provide the article with one or several of the following shapes:

A width H at a transition 27 (see FIG. 2) between the crotch portion 3 and the front portion 2 that is less than the width at the front portion 2. This allows anchoring of the article to/between the thigh muscle tendons of the user and prevents the article from moving backwards during use. In particular embodiments, the width H is in the range 15-45 mm.

A three-dimensional bowl-like shape in an area in the front portion 2. This enhances the body fit.

A ridge-shaped elevation that partially extends between the wearer's buttocks during use of the article. This prevents rearward leakage.

A raised portion (hump) intended to make contact with the genitals of the wearer during use of the article. This provides for better absorption of bodily fluids.

FIGS. 2-3 show absorbent articles including stiffening elements 60, 80 as seen from below with the main purpose of showing examples of alternative geometries of the stiffening element 6. Thus, the lower side 11 of the (upper) absorption member and the lower side 12 of the stiffening element face upwards in these figures. Any lower absorption members are not shown. The position of the absorbent body 5b is indicated with a dashed line.

FIG. 2 shows an absorbent article 100 with a first example of a suitable shape of a stiffening element 60. In this example the stiffening element 60 is flat and has a shape similar to what is shown in EP 1395218. A peripheral edge of the stiffening element 60 is indicated by the reference number 62.

Main features of the stiffening element 60, besides the flatness, are inter alia: i) that it extends in the longitudinal direction of the article 100 over the crotch portion 3 and at least some way in over the front portion 2; ii) that it has a width H at the transition 27 between the crotch and front portions 3, 2 that is within the range of 15-45 mm; iii) that it has a length G in the crotch portion 3 within the range of 70-120 mm; iv) that the side edges of the stiffening element 60, in the direction from the crotch area in over the front portion 2, form an acute angle a (i.e. <90°, preferably)35-55° with a line in the longitudinal direction of the article 100; and v) that it also extends some way in over the rear portion 4 and has a wedge-shaped cutout 66 extending from a rear end edge of the stiffening element 60 in a direction towards the crotch portion 3, as a result of which the product is, during use, imparted with a fold along the longitudinal direction of the article 100 in the cutout 66. The fold extends into the cleft between the buttocks of the wearer during use of the article 100. These features all contribute to the fit of the article to the wearer during use. One or several of these features may be used to enhance the fit of the article. This means, for instance, that the stiffening element 60 does not necessarily extend in over the rear portion 4 and, accordingly, that it can end somewhere in the crotch portion 3 and that there is no cutout 66.

As shown in FIG. 2, the absorption article 100 may be provided with fastening wings 30 provided with adhesives (not shown in FIG. 2) for enhanced attachment of the article to the undergarments of the wearer. Such wings are known.

FIG. 3 shows, in a schematic view, an absorbent article 102 with a second example of a suitable shape of a stiffening element 80. In this case, the stiffening element 80 has the shape of a spoon with a three-dimensional bowl-shaped part located in the front portion 2 and a more narrow part extending longitudinally and centrally over the crotch portion 3, where a hump is formed, and somewhat into the rear portion 4 as to produce a rearward leakage protection as described above.

The invention is not limited by the embodiments described above but can be modified in various ways within the scope of the claims. For instance, the stiffening element 6, 60, 80, irrespective of whether it includes hook material, friction adhesive material or a combination thereof, does not necessarily have to be made entirely of this or these materials. The stiffening element may e.g. include layers of different materials that together build up the total stiffness. The stiffness should be sufficient to give the absorbent article a suitable, predetermined shape during use and that the lower and upper sides 12, 13 of the stiffening element 6, 60, 80 exhibit mechanical fastening properties.

The stiffening element 6, 60, 80 and the upper and lower absorption members 5, 20 may be packaged or sold separately and be assembled by a user prior to use. Either or both of the upper and lower absorption members 5, 20 can be provided with markers for proper positioning of the stiffening element 6. For instance, the lower side 11 of the upper absorption member 5 can be provided with a marker in the form of a line corresponding to the peripheral edge 62 of the stiffening element 6, 60, 80.

The stiffening element 60, 80 can form part of a larger piece of material from which it can be separated, e.g. by cutting. The piece of material can include a marking corresponding to the peripheral edge 62 of the stiffening element 6, 60, 80 for simplifying separation of a stiffening element with proper geometry. Such a piece of material can include several markings corresponding to different sizes of stiffening elements 6, 60, 80.

The stiffening element 6, 60, 80 may be articulated for increasing longitudinal flexibility of the article. “Hinges” for this purpose can be arranged in the form of longitudinally distributed slits or hook-free regions if the piece of material making up the stiffening element includes a hook material.

Foam is an example of a material useful for forming the stiffening element 6, 60, 80.

With reference to FIG. 1, it should be noted that it is not necessary to use a hook material having a single hook carrier layer 9 with hooks 8 protruding in opposite directions. Alternatively, it is possible to use e.g. two parts of a hook material having hooks protruding only in one direction, wherein the parts of the hook material are arranged “back to back” (carrier layer to carrier layer) with the hooks protruding away from the other part of the hook material.

1. A stiffening element for an absorbent article configured to, at least during use of the absorbent article, provide the absorbent article with a predetermined shape that enhances the fit of the absorbent article to a wearer's body, said stiffening element comprising having:
a length in a longitudinal direction and a width in a transverse direction; and
an upper side intended to face towards a wearer during use of the absorbent article and a lower side intended to face away from the wearer during use of the absorbent article,
wherein the stiffening element extends in the longitudinal direction over at least a part of both a front portion and a crotch portion of the absorbent article, and the stiffening element has a width at a transition between the front and crotch portions of the absorbent article that is less than the width in the front portion of the absorbent article, and
wherein at least a part of both the upper side and the lower side of the stiffening element comprises a material exhibiting mechanical fastening properties.

2. The stiffening element according to claim 1, wherein the material exhibiting mechanical fastening properties also contributes to the stiffness of the stiffening element.

3. The stiffening element according to claim 1, wherein the material exhibiting mechanical fastening properties is a hook material having hooks protruding from at least one of the sides of the stiffening element.

4. The stiffening element according to claim 3, wherein the hook material comprises a hook carrier layer to which layer the hooks are secured, and wherein the hook material contributes to the stiffness of the stiffening member.

5. The stiffening element according to claim 4, wherein the hook material constitutes the stiffening element.

6. The stiffening element according to claim 1, wherein the material exhibiting mechanical fastening properties is a friction adhesive material.

7. The stiffening element according to claim 6, wherein the friction adhesive material constitutes the stiffening element.

8. The stiffening element according to claim 1, wherein the stiffening element forms a separate item that is detachably attachable to an absorbent member of the absorbent article.

9. The stiffening element according to claim 1, wherein a width at a transition between the front and crotch portions of the absorbent article is within the range of 15-45 mm.

10. The stiffening element according to claim 1, wherein the stiffening element provides, at least when arranged in association with the absorbent article and during use of said absorbent article, a three-dimensional bowl-like shape in an area in the front portion of the absorbent article.

11. The stiffening element according to claim 1, wherein the stiffening element has a length in the crotch portion of the absorbent article within the range of 70-120 mm.

12. The stiffening element according to claim 1, wherein side edges of the stiffening element, in the direction from the crotch portion of the absorbent article in over the front portion of the absorbent article, form an acute angle a with a line in the longitudinal direction of the stiffening element.

13. The stiffening element according to claim 1, wherein the stiffening element extends further in the longitudinal direction some way in over a rear portion of the absorbent article and has a wedge-shaped cutout extending from a rear end edge of the stiffening element in a direction towards the crotch portion of the absorbent article.

14. The stiffening element according to claim 1, wherein the stiffening element is arranged to allow through-flow of body fluids from its upper side to its lower side.

15. The stiffening element according to claim 1, wherein the stiffening element forms part of a larger piece of material from which it can be separated, and wherein said piece of material comprises a marking corresponding to a peripheral edge of the stiffening element.

16. An absorbent article comprising a stiffening element according to claim 1.

17. The absorbent article according to claim 16, wherein the stiffening element exhibits a stiffness that is higher than a part of the absorbent article that surrounds the stiffening element when the stiffening element is arranged onto the absorbent article.

18. The absorbent article according to claim 16, wherein the absorbent article comprises an absorbent member comprising an absorbent body for absorbing bodily fluids, wherein the absorbent member has an upper side intended to be facing a wearer during use of the absorbent article and a lower side intended to be facing away from the wearer during use of the absorbent article, and wherein the stiffening element is detachably attachable to the lower side of the absorbent member.
